(19) United States
(12) Patent Application Publication
(10) Pub. No.: US 2023/0307128 A1
(43) Pub. Date: Sep. 28, 2023

(54) METHODS FOR ASSESSING BRAIN HEALTH USING BEHAVIOURAL AND/OR ELECTROPHYSIOLOGICAL MEASURES OF VISUAL PROCESSING

(71) Applicant: BAYCREST CENTRE FOR GERIATRIC CARE, TORONTO (CA)

(72) Inventors: ALLISON SEKULER, NORTH YORK (CA); EUGENIE ROUDAIA, NORTH YORK (CA); ALI HASHEMI, NORTH YORK (CA)

(21) Appl. No.: 18/011,339

(22) PCT Filed: Jun. 21, 2021
(86) PCT No.: PCT/CA2021/050847

§ 371 Date: Dec. 19, 2022

Related U.S. Application Data
(60) Provisional application No. 63/041,666, filed on Jun. 19, 2020.

(51) Int. Cl. G16H 50/20; A61B 5/16

(52) U.S. Cl. CPC G16H 50/20; A61B 5/165; A61B 5/7264

(57) ABSTRACT

The present invention provides a method for assessing brain health in a subject, comprising the steps of administering one or more visual perception tests to the subject; obtaining a measurement of behavioural responses during the one or more visual perceptual tests and extracting indices of perceptual ability for each test from the behavioural responses; and/or obtaining a measurement of electrophysiological responses of the subject during administration of the one or more visual perception tests, and extracting indices from the electrophysiological responses for each task, wherein a time-based correlation of events occurring during the tests with the obtained electrophysiological responses is also obtained; and comparing the obtained behavioural indices, the obtained electrophysiological indices, or both with a comparative data set of healthy older adults with normal cognition, or a historical data set of the same individual, to identify deviations from normative or historical performance in any measure to provide an assessment of brain health in the subject.

### FIELD OF THE INVENTION

The present invention pertains to the field of age-related neurodegenerative disorders and in particular to the screening, assessment, or diagnosis of same.

### BACKGROUND

Neurodegenerative disorders linked with aging begin to affect the brain many years before individuals experience symptoms. As a result, Alzheimer's disease (AD) and other related dementias are diagnosed much too late, when the neural damage is already too advanced to allow for effective treatment. The main problem this invention is addressing is the challenge of assessing brain health in older adults for the purposes of detecting the presence of neurodegenerative diseases at early or preclinical stages.

Biological markers of neurodegeneration, such as tau and amyloid beta, can be used to identify individuals at risk of AD during preclinical stages of the disease, but these biomarkers require expensive and sometimes invasive procedures. Alternatively, neuropsychological paper and pencil or computer-based testing can be used to diagnose brain diseases, but many of these tests suffer from several disadvantages. First, they require trained experts to be administered, they take a very long time (often up to 3-4 hours), they are not sensitive to subtle changes in brain function, and they have difficulty discriminating among different types of disease aetiologies. Finally, declines in performance in the various neuropsychological tests are difficult to relate to specific brain functions, as performance is often affected by motor ability, visual perception, attention, language, and/or cultural barriers.

While memory loss is one of the most known symptoms of Alzheimer's disease, visual perception and attention are known to be affected at very early stages of AD. However, these changes may be less noticeable to the individual at early stages, and existing neuropsychological tools do not evaluate visual perceptual function and attention in the optimal way. For example, tests of visuospatial perception in neuropsychological batteries often require participants to draw or copy figures or to name visual objects. These response strategies may lead to motor ability or language playing a disproportionate role in performance on these visuospatial tests.

The Montreal Cognitive Assessment (MoCA) is the most common screening tool that is used to identify individuals at risk of Alzheimer's disease (Nasreddine et al., 2005). Although the MoCA requires minimal training to administer, and consists of several tests that evaluate visual perception, language, memory, abstract thinking, one of the problems associated with this test is that performance on the MoCA score depends on the level of education and linguistic ability of the participant. Furthermore, the MoCA does not always identify individuals at earliest stages of disease progression.

Cogniciti is another screening test that uses visual perceptual, memory, and executive function tasks to evaluate brain health (Troyer et al., 2014). While it is simple, quick, and is self-administered, most of its tasks evaluate higher-order cognitive and executive function, which may be impacted later in the time-course of the disease relative to visual perceptual functions.

Therefore there is a need for a method to assess brain health in older adults for the purposes of detecting signs of pathological brain aging caused by the presence of a neurodegenerative disorder, and that does not rely on linguistic abilities, memory, or specific cultural knowledge, and that can be administered in a primary care setting or in the community by non-trained experts using easy to access tools or technologies.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide methods for assessing brain health using behavioural and electrophysiological measures of visual processing. In accordance with an aspect of the present invention, there is provided a method for assessing brain health in a subject, comprising the steps of: administering one or more visual perception tests to the subject; obtaining a measurement of behavioural responses during administration of the one or more visual perceptual tests and extracting indices of perceptual ability for each test from the behavioural responses; and/or obtaining electrophysiological measurements of the subject during administration of the one or more visual perception tests, and extracting indices from the electrophysiological measurements for each task, wherein a time-based correlation of events occurring during the tests with the obtained electrophysiological measurements is also obtained; and comparing the obtained behavioural indices, the obtained electrophysiological indices, or both with a comparative data set of normative data set of healthy older adults with normal cognition, or a historical data set of the same individual, to identify deviations from normative or historical performance in any measure to provide an assessment of brain health in the subject.

### BRIEF DESCRIPTION OF THE FIGURES

FIG. 1A is a schematic depiction of the steps in the Contours in Clutter test, in accordance with one implementation of the present invention. FIG. 1B is an example of a stimulus screen.

FIGS. 1C-F are graphical representations of data obtained using the Contours in Clutter test for subjects having NC and MCI.

FIG. 2A is a schematic depiction of the steps in the Face Identification test, in accordance with one implementation of the present invention.

FIGS. 2B-E are graphical representations of data obtained using the Face Identification test for subjects having NC and MCI.

FIG. 3A is a schematic depiction of the steps in the Emotion Recognition in Biological Motion test, in accordance with one implementation of the present invention.

FIGS. 3B-E are graphical representations of data obtained using the Emotion Recognition in Biological Motion test for subjects having NC and MCI.

FIGS. 4A-D are graphical representations of data obtained from assessment of four standardised vision measures for subjects having NC and MCI.

FIG. 5A is a graphical representation of the change in the relative density of the stimulus in one subject's block of trials in the Contours in Clutter test, and FIG. 5B is a graphical representation of the corresponding accuracy values at each density level, and a best-fit psychometric function used to estimate the density threshold.

FIGS. 6A-C are schematic depictions of the steps in the Central and Peripheral Divided Attention test, in accordance with one implementation of the present invention.

FIGS. 7 and 8A-D are graphical representations of data obtained using the Central and Peripheral Divided Attention test for subjects having NC and MCI.

FIG. 9 is a graphical representation of the results of a multivariate discriminant analysis combining behavioural and EEG outcome measures across all the tests.

### DETAILED DESCRIPTION OF THE INVENTION

As used herein, the term “about” refers to a +/−10% variation from the nominal value. It is to be understood that such a variation is always included in a given value provided herein, whether or not it is specifically referred to.

Unless defined otherwise, all technical and scientific terms used herein have the same meaning as commonly understood by one of ordinary skill in the art to which this invention belongs.

Brain changes linked with Alzheimer's Disease (AD) begin years before the onset of clinical symptoms. The difficulty in identifying individuals during preclinical stages of Alzheimer's disease or other neurodegenerative disorders is a significant barrier to implementing interventions to prevent dementia. Simple, visual tasks that do not rely on linguistic ability or memory, paired with sparse electroencephalography (EEG), may provide viable markers for identifying individuals at risk of dementia due to underlying neurodegenerative disorders, such as Alzheimer's disease, Lewy body disease, fronto-temporal dementia, or vascular pathology.

The present invention provides a quick, affordable method to assess brain health in older adults for the purposes of detecting the presence of a neurodegenerative disorder at preclinical or clinical stages, and that does not rely on linguistic abilities, memory, or specific cultural knowledge, and that can be administered in a primary care setting or in the community by non-trained experts.

The present invention also provides a framework for monitoring brain health over time, for example, to track progression of disease, effects of treatments or behavioural interventions, or to predict perceptual and cognitive status changes.

The visual perceptual and attentional tests employed in the present methods are designed to assess specific visual perceptual functions, while requiring minimal language, memory, and motor skills. The use of psychophysical procedures that adapt to the user's performance to estimate a stimulus parameter value that yields a certain level of performance safeguards the tests from ceiling/floor effects and allows them to be flexibly used with individuals across a range of abilities. The experimental stimuli can therefore be standardized and controlled to avoid practice effects from retesting, thus improving the reliability and reproducibility of the assessments.

Furthermore, the methods of assessment employ tests that do not require a trained professional to administer or score, as data collection and processing is controlled by an algorithm and test administration is performed by a computer or mobile device. Language skills, education level, or cultural differences do not impact performance in the tests, because the tests are designed to be very simple to understand and depend only on specific visual perceptual and attentional functions tested. Finally, the addition of sparse EEG recorded simultaneously can provide a measure of the efficiency and speed of neural processes related to perceptual processing. These neural markers may be important especially for detecting cognitive decline at early stages, by detecting recruitment of compensatory mechanisms that may mask any differences in behavioural performance (McIntosh et al., 1999).

This invention provides an integrated set of visual perceptual tests that are selected to probe an array of different functions known to be affected by neurodegenerative diseases, such as Alzheimer's, or known to rely on neural structures that are differentially affected in various neurodegenerative disorders. Considering that visual and attentional functions may be one of the earliest symptoms to be affected in many individuals with Alzheimer's disease and other neurodegenerative disorders, a set of easy to administer, sensitive tests can play an important role in identifying individuals at risk of dementia earlier and at a larger scale (e.g., in the community).

The present invention, in one embodiment, provides a method for assessing brain health in a subject, comprising the steps of administering one or more visual perception tests to the subject, and obtaining a measurement of behavioural responses during administration of the one or more visual perceptual tests and extracting indices of perceptual ability for each test from the behavioural responses; and/or obtaining a measurement of electrophysiological responses of the subject during administration of the one or more visual perception tests, and extracting indices from the electrophysiological responses for each task, wherein a time-based correlation of events occurring during the tests with the obtained electrophysiological responses is also obtained; and comparing the obtained behavioural indices, the obtained electrophysiological indices, or both with a comparative data set of healthy older adults with normal cognition, or a historical data set of the same individual, to identify deviations from normative or historical performance in any measure to provide an assessment of brain health in the subject.

In accordance with the present invention, the visual perceptual tests that may be employed to carry out the assessment methods include a Contour in Clutter test, a Face Identification test, an Emotion Recognition in Biological Motion test, and a Central and Peripheral Divided Attention Test.

This set of tests is designed to provide an assessment of distinct, non-overlapping visual perceptual functions that each can index unhealthy brain aging independently. By examining the pattern of performance across the different tests, it may also be possible differentiate amongst different types of neurodegenerative disorders earlier that can currently be achieved using known assessment regimes.

Accordingly, in one embodiment, the methods of the present invention can employ the administration of a subset or all of the visual perceptual tests described above, which evaluate intermediate-stage processing in spatial vision, face processing, emotion recognition in biological motion, and visual processing speed and ability to extract information across a wide field of view.

In one embodiment, the visual perception test comprises one or more trials, wherein a trial comprises the steps of presenting a visual stimulus to the subject, and recording a behavioural response by the subject and/or recording an EEG response to brain activity during the trial.

In a preferred embodiment, the measurement of behavioural responses during the visual perceptual tests is carried out simultaneously with measurement of EEG responses.

In a preferred embodiment, the step of measuring electrophysiological responses comprises monitoring EEG responses of the subject. In one embodiment, the measurement of the electrophysiological responses is carried out using a portable EEG device.

In one embodiment, using the methods of the present invention, an individual's performance on these tests can be compared to previously collected data sets of individuals with a range of cognitive diagnoses, from normal cognition to dementia, due to different aetiologies such as Alzheimer's disease with posterior cortical atrophy, typical Alzheimer's presentation, Lewy Body disease, or normal aging, to assist in determining the presence, stage, and type or subtype of a neurodegenerative disease.

Accordingly, in one embodiment, the comparative data set is a normative data set of healthy older adults with normal cognition to identify deviations from normative performance in any measure or combinations of measures to provide an assessment of brain health in the subject.

In one embodiment, the assessment of brain health comprises determination of mild cognitive impairment.

In one embodiment, the assessment of brain health comprises determination of a neurodegenerative disorder, wherein the neurodegenerative disorder is classical Alzheimer's disease, Alzheimer's disease with posterior cortical atrophy, Lewy Body disease, frontotemporal dementia, or vascular dementia.

In another embodiment, an individual's current performance can be compared to previous performance across some or all tasks to visualize change in visual perceptual abilities across a range of tests. Accordingly, this invention can also be used to track performance across time to monitor one's brain health in relation to treatment (e.g., neurostimulation, new medication, chemotherapy), behavioural interventions (exercise program, diet, cognitive training), etc. By comparing an individual's data with a normative data set of longitudinal changes, it can detect unusual changes in specific brain functions before overt clinical symptoms occur.

Accordingly, in one embodiment, the assessment of brain health comprises an assessment of changes in brain health over a period of time to track progression of disease, to track effects of treatments or behavioural interventions, or to predict changes in perceptual and cognitive status.

Accordingly, in one embodiment, the comparative data set is data obtained in one or more tests previously administered to the subject, to identify changes in performance in any measure or combinations of measures to provide an assessment of changes in the brain health state in the subject.

The invention will now be described with reference to specific examples. It will be understood that the following examples are intended to describe embodiments of the invention and are not intended to limit the invention in any way.

### Examples

### Apparatus

All experimental tests were programmed in Matiab (The Mathworks Inc., USA) using the Psychophysics toolbox, driven by a PC computer running Ubuntu 16.04. Visual stimuli were presented on a 33.9 cm×27.1 cm cathode-ray tube monitor with a 1280×1024 resolution and a refresh rate of 85 Hz. The monitor display was the only light source in the room and had an average background luminance of 55.1 cd/m². Participants viewed the display binocularly from a viewing distance of 114 cm. Participant responses were acquired using a MilliKey MH-6 response box (LabHackers Inc., Ottawa, Canada).

The Muse™ 2016 Headband (InteraXon, Ontario, Canada), a battery-powered EEG headband containing four dry electrodes at TP9, AF7, AF8 and TP10 (10-20 electrode system) and a reference electrode at Fpz, was used to record EEG data. All data from the Muse™ 2016 was streamed via Bluetooth to an EEG recording laptop running Ubuntu 16.04. The LabStreamingLayer software (https://github.com/sccn/labstreaminglayer) was used to synchronize time-stamped event markers from Matlab with EEG sensor data.

### General Procedure:

Prior to the start of each task, a text display presents the instructions for each task and the experimenter explains the task instructions to the participant. Then, several example trials are shown to ensure participants understand the instructions, followed by several practice trials comprising stimuli at an easy difficulty level. Practice trials may be repeated until the participant performs with a high accuracy at the easy level. When the experimental trials begin, each trial begins with a fixation point shown against a blank screen and the participant is asked to fixate on the fixation point. After that, the stimulus appears for a fixed stimulus presentation duration, followed by a response screen, which remains on the screen until a response is made using a response button or another method such as a verbal response. Once a response is given, the next trial continues immediately after. After each trial, the experimenter has the option to disregard that trial if they observe that the participant was not looking at the screen during the trial presentation. The experimenter also has the option to switch to a ‘slow mode’ that requires the experimenter to press the space bar to start each trial. This mode is necessary when testing individuals at more advanced stages of cognitive impairment, whose attention span may not allow them to maintain focus for the duration of the task. This mode also allows the experimenter to remind participants about the task requirements if necessary. Finally, each test begins with a series of easy trials before progressively becoming more difficult.

### Tests:

### 1. Contours in Clutter Test

In one embodiment, the visual perception test is a Contours in Clutter test, wherein the visual stimulus comprises a sequence of images, each image comprising an array of oriented visual stimulus elements in which a subset of stimulus elements forms a target contour through alignment of neighboring elements. The visibility of the target contour is varied between trials by altering the display characteristics to measure an ability of the subject to perceive the target contour within a cluttered background, wherein the subject identifies an element of the target contour to exhibit knowledge of the shape and/or location of the definable contour.

In one embodiment, a measure of the subject's tolerance for clutter in performing the task and/or the EEG response is compared with the comparative data set, in isolation or in combination with other measures.

In one embodiment, the degree of clutter is varied across trials to determine the degree of clutter that yields a specified level of accuracy.

In one embodiment, wherein the target contour is a spiral-shaped contour formed through alignment of the orientations of the subset of stimulus elements along a spiral path, and the subject is prompted to report whether the tail of the spiral-shaped contour is on the left or right side of the image.

In one embodiment of the Contours in Clutter test, the visual stimulus comprises a sequence of images, each image comprising an array of randomly oriented elements comprising background elements and a subset of contour elements arranged to form a spiral-shaped contour through alignment of the orientations of the subset of contour elements along the spiral path, and the subject is prompted to report whether the tail of the spiral-shaped contour was on the left or right side of the image. In this embodiment, the relative density of the background and contour elements used in each image in the sequence of images is varied to determine the degree of relative density that yields a specified level of accuracy.

In one embodiment, an EEG response is acquired during performance of the Contours in Clutter test.

In one embodiment, the degree of relative density to yield a specific level of accuracy and/or the EEG response is compared with the comparative data set, in isolation or in combination with other measures.

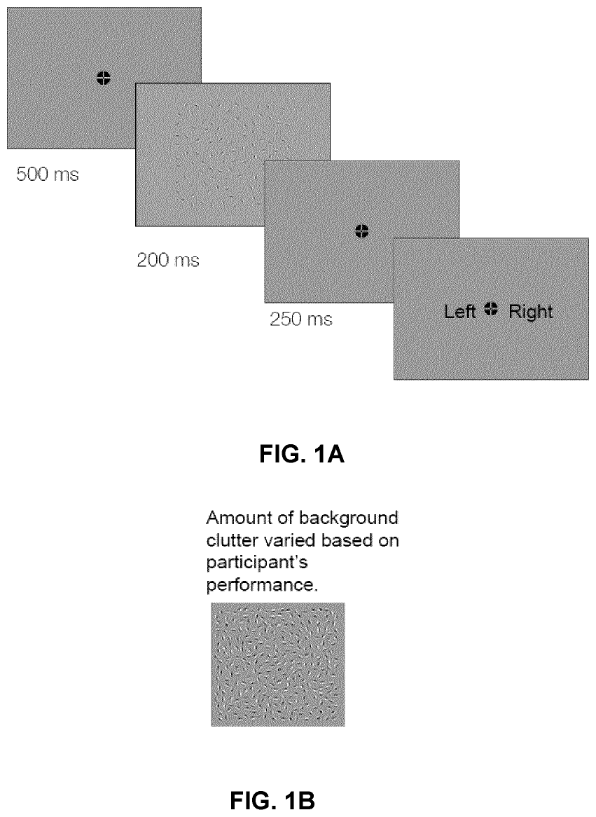

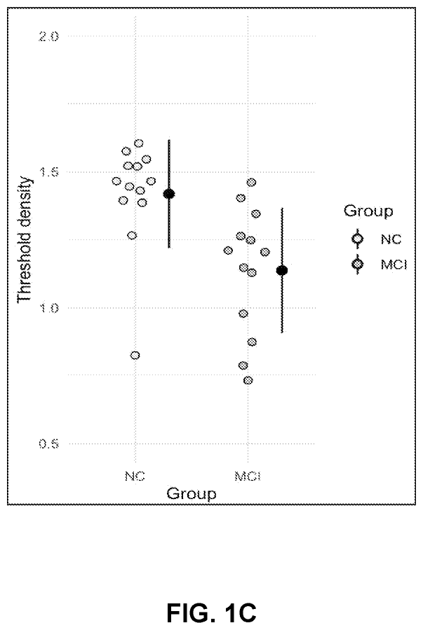

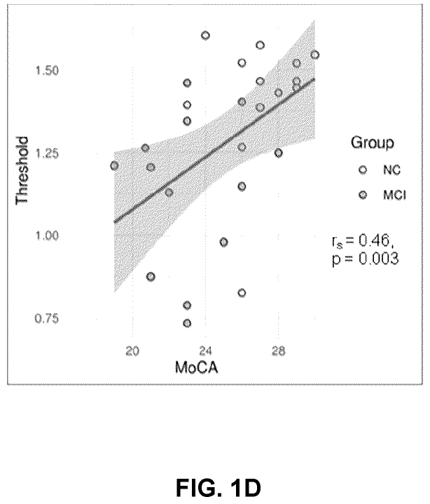

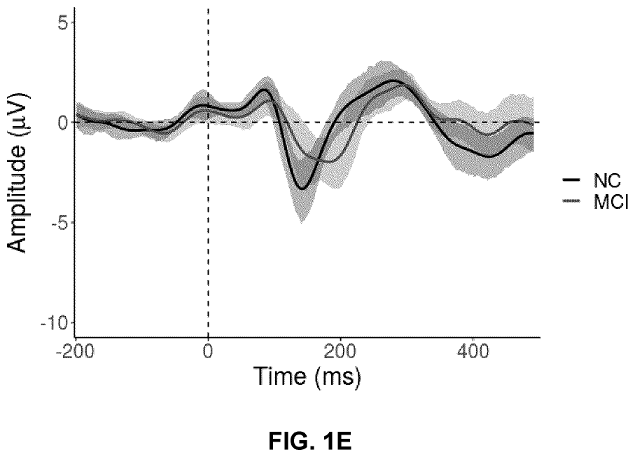

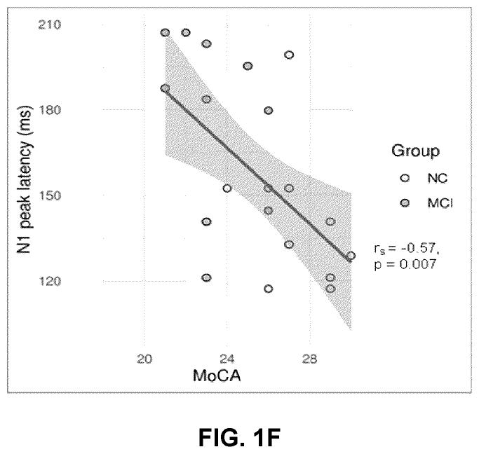

FIG. 1A is a schematic depiction of the steps of one embodiment of the Contours in Clutter Test and FIG. 1B is an exemplary stimulus screen used in this test. In the test, stimuli comprise an array of randomly oriented elements (Gabors) (Roudaia et al., 2013). A subset of the Gabors are placed on a spiral-shaped contour path, and the individual Gabor orientations are aligned along the spiral path. The location of the spiral contour within the array and its global orientation is varied across trials. On each trial, the stimulus is shown briefly (˜200 ms), followed by a blank screen. Then participants are presented with a prompt screen that asks to report the global orientation of the contour. Specifically, participants are asked to report whether the tail of the spiral was on the left or right side of the display by pressing one of two buttons.

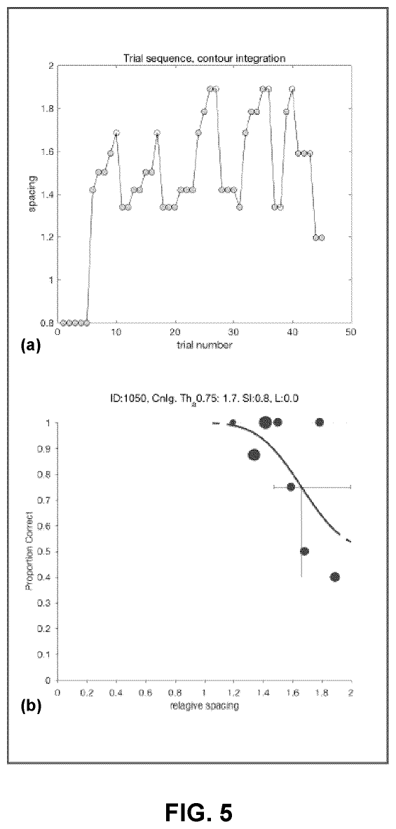

FIG. 5A is a graphical representation of one subject's block of trials in the Contours in Clutter Test, where the y-axis corresponds to the value of relative spacing of the background and contour elements used on each successive trial. Across trials, the relative spacing of contour and background elements varies according to an adaptive procedure, QUEST+(Watson, 2017), to determine the maximum relative background-contour density at which the participant can report the global orientation of the spiral with 75% accuracy. FIG. 5B is a graphical representation of the accuracy values as a function of relative spacing obtained during a single block (blue dots, whose size is proportional to the number of trials at that stimulus level), the best-fit psychometric function (blue thick line), and the estimate of the 75% correct threshold and its confidence interval (vertical line and blue error bars). This relative density threshold reflects the ability of the visual system to group orientation information across space to extract a contour while segregating it from surrounding clutter. The test ends when the threshold is estimated with a specified precision, or after a fixed number of trials (e.g., 45). This test lasts approximately 6 minutes.

Performance in this task is dependent on interactions between early visual areas, the lateral occipital cortex, and the inferior parietal lobule (Hess et al., 2003; Volberg & Greenlee, 2014). Previous studies using a similar task found impaired performance only in individuals with AD who had occipital pathology and preserved thresholds in other AD participants, nor in mild cognitive impairment (MCI) (Uhlhaas et al., 2008). However, unlike in the current test, Uhlhaas et al.'s task allowed a very long inspection time (1 min), which may have reduced the ability to detect subtle impairments in the MCI stage. Moreover, EEG markers related to contour integration have not been previously examined in older adults with MCI or dementia, but their temporal profiles may be impacted by early changes in the inferior parietal lobule.

### 2. Face Identification Test

In one embodiment, the visual perception test is a Face Identification test, wherein the visual stimulus comprises a sequence of images, the sequence of images comprising a first image and a second image, wherein the first image is a test face, and the second image comprises two or more probe faces, wherein one of the probe faces is of the same identity as the test face, and wherein the subject identifies which probe face matches the identity of the test face.

In one embodiment, the difficulty of the task is varied across trials by altering the display characteristics to measure the ability of the subject to identify face identity under different stimulus conditions, wherein the display characteristics are altered by varying one or more of the stimulus duration, stimulus contrast, degree of added visual noise, manipulation of facial appearance, facial viewpoint, facial orientation, addition of a masking stimulus before or after presentation of faces.

In one embodiment of the Face Identification test, the visual stimulus comprises a sequence of images, the sequence of images comprising a first image and a second image, wherein the first image is a test face facing in a first direction, and the second image comprises two or more probe faces, wherein each probe face is facing in a second direction different from the first direction, and wherein one of the probe faces shows the same person as the test face, and the subject is prompted to indicate which probe face matches the identity of the test face.

In one embodiment, an EEG response is acquired during performance of the Face Identification test.

In one embodiment, a measure of the ability of the subject to identify face identity when performing the task and/or the EEG response is compared with the comparative data set, in isolation or in combination with other measures.

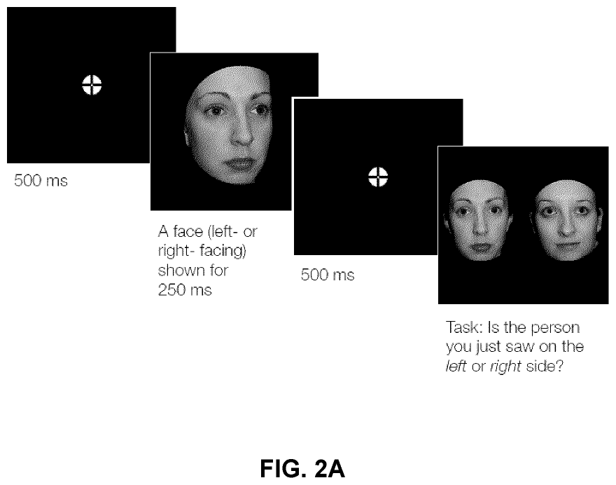

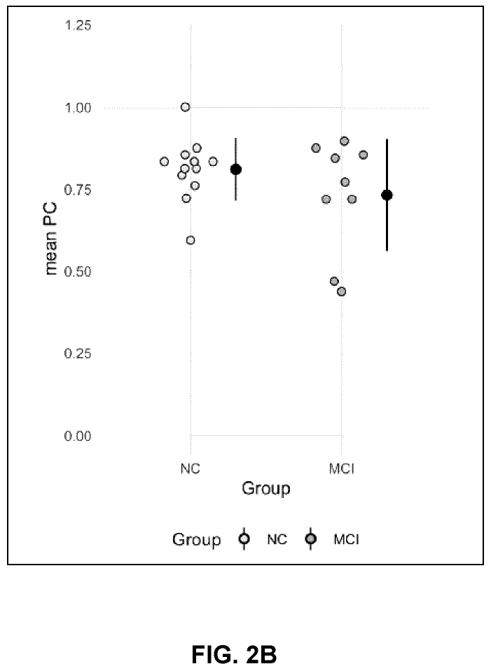

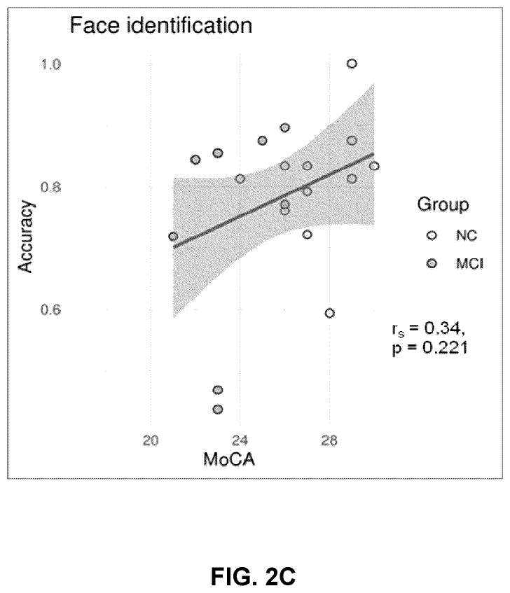

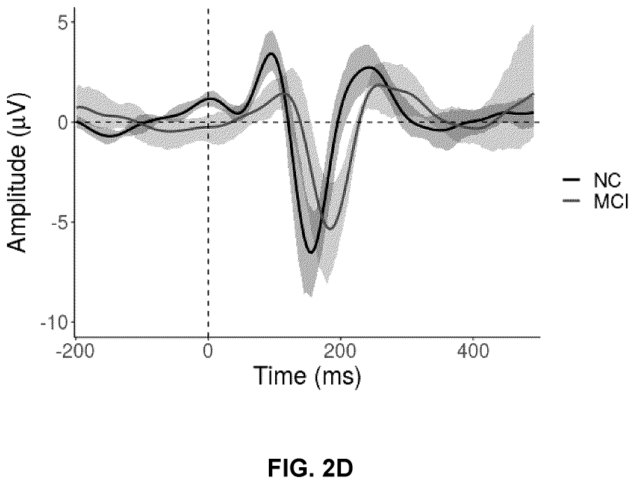

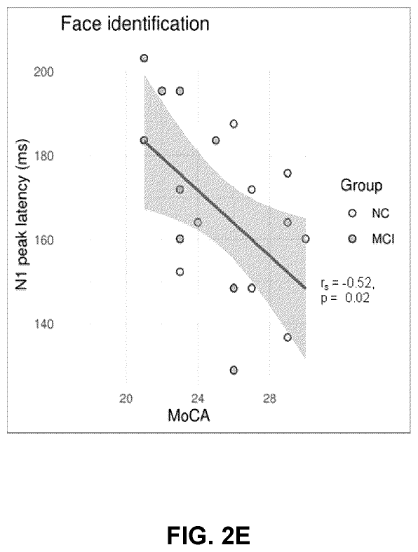

FIG. 2A is a schematic depiction of the steps of one embodiment of the Face Identification test (Konar et al., 2013). In this task, on each trial, a grayscale picture of a face (test face) that is facing slightly left or right of centre is shown briefly in the middle of the screen.

Shortly after the test face disappears, a prompt screen is shown, containing two or more probe face pictures, all facing in the same direction, such as in frontal view. The participant is asked to indicate which probe face matches the identity of the face shown previously by pressing a button as quickly and accurately as possible. The slight change in viewpoint across the test and probe images ensures that participants cannot do the task by matching a single point on the image, but rather have to make the judgement based on the face identity. Across trials, different face identities are shown in the test phase. On the probe screen, the location of the same-identity face is selected at random among the possible locations. The different-identity faces change across trials, but are restricted to match the gender and ethnicity of the test face, to ensure that the task cannot be done based on information other than face identity. The accuracy of the response is measured. The test lasts approximately 5 minutes.

This face identification task is included to probe high-level processing of the ventral stream, as face identification is mediated by areas in the inferior temporal cortex. It has been suggested that face identification is specifically impaired in AD beyond any deficits in facial recognition associated with memory declines (Lavallée et al., 2016). A recent study found that individuals with amnestic MCI (aMCI) showed delays in the event-related potential (ERP) response to face stimuli (Yamasaki et al., 2016), even while behavioural performance was preserved.

### 3. Emotion Recognition in Biological Motion Test:

In one embodiment, the visual perception test is an Emotion Recognition in Biological Motion test, wherein the visual stimulus comprises a sequence of animated images, each animated image comprising depictions of a human walker conveying an emotional state through its gait, and the subject indicates which emotion the walker portrayed.

In one embodiment, the difficulty of the task is varied across trials by altering the display characteristics to measure the ability of the subject to identify the emotion of the walker under different stimulus conditions, wherein the display characteristics are altered by varying one or more of the stimulus duration, stimulus contrast, degree of added visual noise, manipulation of walker appearance, walker viewpoint, walker orientation, addition of a masking stimulus before or after presentation of walkers.

In one embodiment of the Emotion Recognition in Biological Motion test, the visual stimulus comprises a sequence of animated images, each animated image comprising depictions of point light walker shown for a single gait cycle, wherein the gait is happy, angry, or sad; and the subject is prompted to report which emotion the point light walker portrayed.

In one embodiment, an EEG response is acquired during performance of the Emotion Recognition in Biological Motion test.

In one embodiment, a measure of the ability of the subject to identify emotion of the walker when performing the task and/or the EEG response is compared with the comparative data set, in isolation or in combination with other measures.

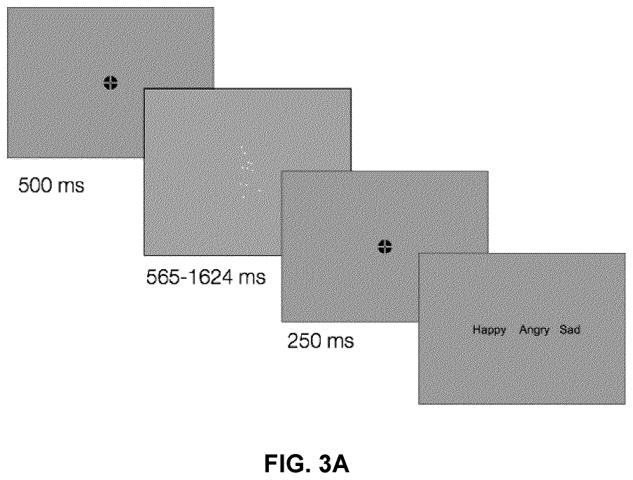

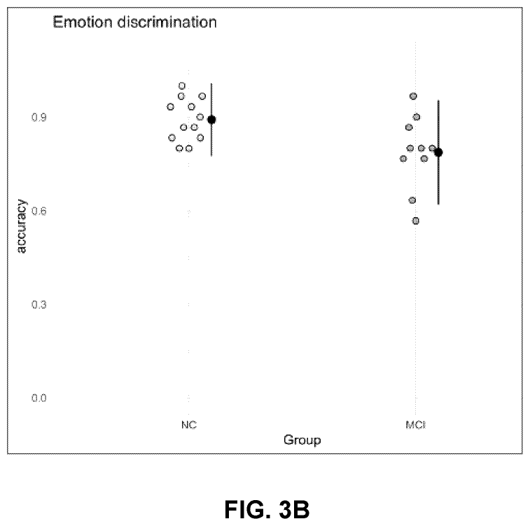

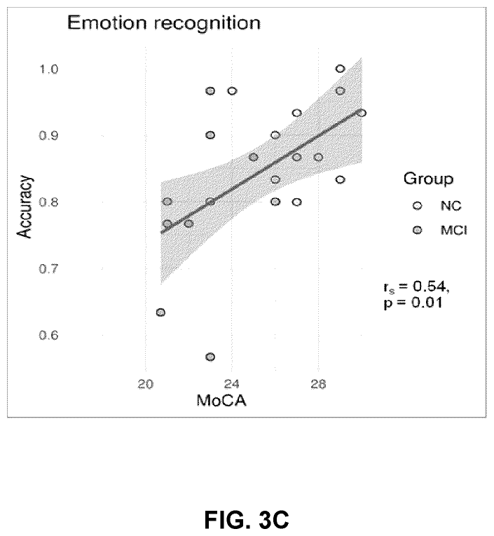

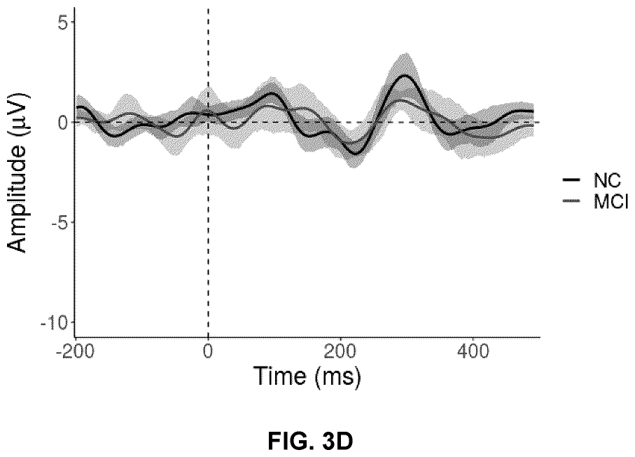

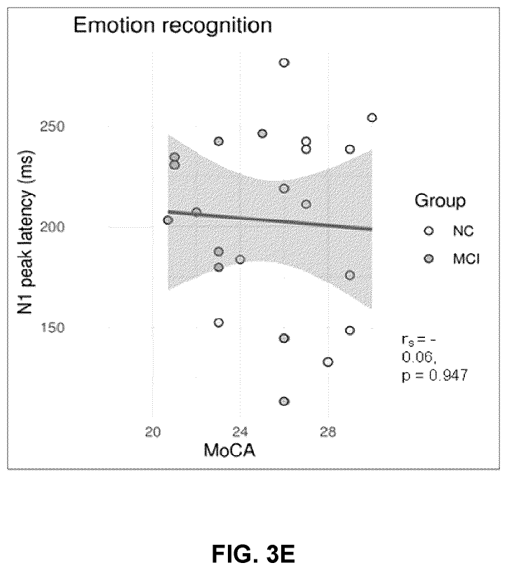

FIG. 3A is a schematic depiction of the steps of one embodiment of the Emotion Recognition in Biological Motion test. Following (Spencer et al., 2016), in this task, a point light walker is shown for a single gait cycle in the middle of the display for ˜2 s. The walker is either happy, angry, or sad. Optionally noise dots can be presented in conjunction with the walker. Point light walker motion is taken from several different walkers with various emotions and each one is repeated several times in random order. When the prompt screen is shown, participants are asked to report which emotion the point light walker portrayed. Performance is measured using accuracy. This task takes approximately 5 minutes.

Perception of biological motion requires the integration of form and motion information and recruits a wide network of association areas, including the superior temporal sulcus (Peuskens et al., 2005). Processing the emotional content of biological motion involves areas beyond the superior temporal sulcus to include the frontal gyrus and in the anterior temporal pole (Jastorff et al., 2016). Interestingly, patients with a behavioural variant of frontotemporal dementia (FTD) showed a specific deficit in processing emotion in point-light-walkers relative to AD and LBD (Jastorff et al., 2016). Thus, the perception of emotion in biological point light walkers may be impaired in individuals at pre-clinical stages of FTD.

### 4. Central and Peripheral Divided Attention Test

This test evaluates the ability to quickly extract information across the visual field under focused and divided attention conditions (Sekuler et al., 2000).

In one embodiment, the visual perception test is a Central and Peripheral Divided Attention test, wherein the visual stimulus comprises a sequence of images, each image comprising one or more central target(s) and/or one or more peripheral target(s) presented in a number of possible locations; and wherein the subject reports target attributes selected from the identity of the central target(s), or one or more of the radial direction, location, identity of the peripheral target(s), or both central and peripheral target attributes.

In one embodiment, the difficulty of the task is varied across trials by altering the display characteristics to measure the ability of the subject to identify target attributes in central and/peripheral locations, wherein the display characteristics are altered by varying one or more of the stimulus duration, stimulus contrast, spatial distribution of target locations, degree of added visual noise, numbers of central and/or peripheral targets, similarity among central and/or peripheral targets, addition of a masking stimulus before or after presentation of targets.

In one embodiment, the visual perception test is a Central and Peripheral Divided Attention test, wherein the visual stimulus comprises a sequence of images, each image comprising a central target and/or a peripheral target presented in one of a number of possible locations; wherein the subject is cued in advance of a trial whether the trial will comprise a central target, a peripheral target, or both targets; wherein the duration of target exposure is varied across trials to determine a target exposure duration that yields a specified accuracy level; and wherein, the subject is asked to report the identity of the central target, or to report the radial direction of the peripheral target, or to report the attributes of both targets.

In one embodiment, at the start of a trial, a preparatory screen comprising a central fixation point and 16 circular outlines is presented to the subject, wherein the central fixation point indicates the location of the central target and the circular outlines indicate the potential locations of the peripheral target. If a subsequent central target appears, the subsequent central target is selected from among a set of letters, and if a subsequent peripheral target appears, the subsequent peripheral target is a filled circle within one of the 16 circular outlines. In addition, subsequent to presentation of the central and/or peripheral target, all potential target locations are covered with a masking stimulus.

In one embodiment, an EEG response is acquired during performance of the Central and Peripheral Divided Attention test.

In one embodiment, a measure of the ability of the subject to identify the target attributes when performing the task and/or the EEG response is compared with the comparative data set, in isolation or in combination with other measures.

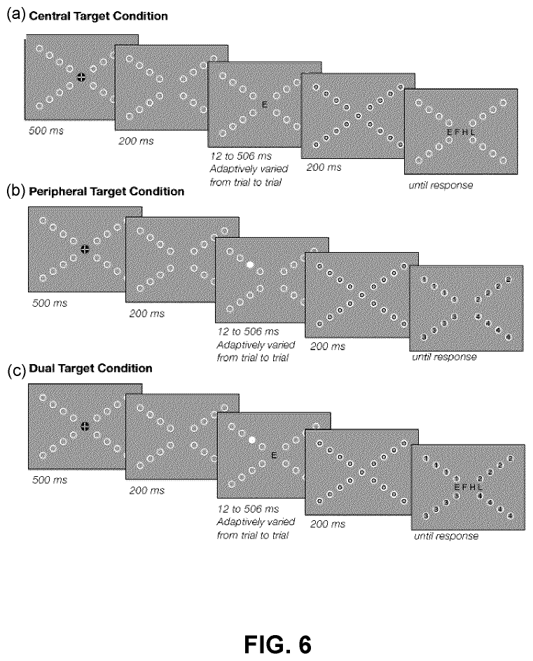

FIGS. 6A-C are schematic depictions of the three conditions in one embodiment of the Central and Peripheral Divided Attention test and all the steps in each condition. The stimuli comprise an array of 16 white circular shapes of 1.3° diameter each, all equally spaced along four radial spokes forming an X-like configuration, placed at 4°, 8°, 12° and 16° in the periphery, against a uniform grey background. This array of circles indicates the possible locations of the peripheral target. The peripheral target comprises a white, filled circle with a diameter of 1° that can appear inside any one of the 16 circular shapes. The central target is a single white letter randomly chosen among a pool of four letters (E, F, H and L) and measuring ˜1.0°. A masking display comprises high contrast checkerboards measuring 1.3°×1.3° presented on the same locations as the 16 circular shapes and the central target.

First, participants complete the Central target, Focused Attention condition (FIG. 6A). In this condition, participants see a display with a fixation point in the centre and the X-like array of 16 circular outlines. Participants are asked to fixate on a central fixation point and monitor this location for a central target. After 500 ms, the fixation point disappears for 200 ms and the central target is flashed briefly, followed by the masking display for 500 ms. Finally, a reponse screen appears showing the letters E, F, H, L in the centre of the screen, and the 16 circular outlines. Participants are asked to report which letter they saw. Auditory feedback is provided in the form of a high-pitched tone to indicate a correct response or a low-pitched tone to indicate an incorrect response. Across trials, the duration of presentation of the central target is varied by the Quest+ algorithm to estimate a duration threshold. In the current experiment, participants completed 30 trials in this condition.

Next, participants complete the Peripheral Target, Focused Attention condition (FIG. 6B), At the start of each trial, the same X-like array of 16 circular outlines and a central fixation point is shown. Participants are asked to fixate the fixation point and to monitor the display for the peripheral target. After the fixation point disappears, a peripheral target is flashed in one of 16 circular outlines for a brief duration. The masking display is then shown for 500 ms, after which the response screen appears, in which each ring is labeled with the number of its spoke (1, 2, 3, or 4). Participants are asked to report which of the four spokes containing the peripheral target. Auditory feedback is provided in the form of a high-pitched tone to indicate a correct response or a low-pitched tone to indicate an incorrect response. Across trials, the duration of presentation of the peripheral target is varied by the Quest+ algorithm to estimate a duration threshold. In the current experiment, participants completed 45 trials in this condition.

Next, participants complete the Divided Attention condition (FIG. 6C). At the start of each trial, the same X-like array of 16 circular outlines and a central fixation point is shown. Participants are asked to fixate the central fixation point and to monitor the display for the central target and the peripheral target, which will appear at the same time. After the fixation point disappears, the central target and a peripheral target are shown simultaneously, for the same duration. The masking display is then shown for 500 ms. The response screen then appears, comprising the 16 circular outlines each labeled with its spoke number (1, 2, 3, or 4) and the four possible letters (E, H, F, L) shown in the centre of the display. Participants are asked to report which letter was the central target and which spoke contained the peripheral target. Auditory feedback is provided for each response consecutively, with a high-pitched and low-pitched tone indicating a correct and an incorrect response, respectively. Across trials, the duration of presentation of the two targets is determined by one of two Quest+ procedures selected at random. One Quest+ procedure is estimating the duration threshold for the central target, and the other procedure is estimating the duration threshold of the peripheral target. Each Quest+ procedure is updated with the duration of presentation used on each trial and the accuracy of the response for its relevant target. In the current experiment, participants completed 45 trials in this condition.

It is known that older adults show a greater increase in duration thresholds in the divided attention condition, relative to the focused attention conditions (Sekuler, Bennett, & Mamelak, 2000). There is also some evidence that dividing attention may be even more difficult for individuals with Alzheimer's disease and MCI (Mapstone et al., 2008; Rizzo, Anderson, Dawson, Myers, & Ball, 2000b, Okonkwo et al., 2008). However, no prior studies had examined performance for the central and peripheral targets separately in the context of aging and neurodegeneration.

### Standardised Vision Measures

The following standardized vision measures are carried out to ensure that the participants are suitable candidates. Measurement of their visual acuity and contrast sensitivity can provide confidence that performance in the behavioural tasks is not limited due to an inability to actually see the stimuli precisely, but limited due to processes in the brain. Further, measures of acuity and contrast sensitivity can be used as covariates to improve the predictive models.

### Visual Acuity (Near and Far)

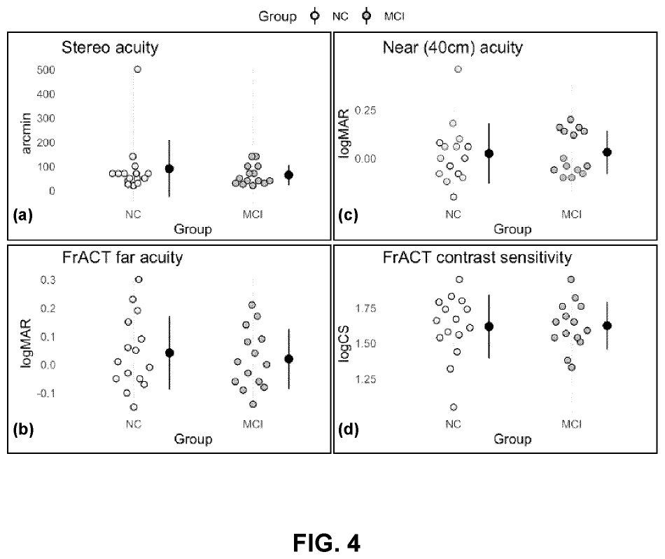

Far visual acuity is measured with the computerized Freiburg Visual Acuity and Contrast Test (FrACT) (Bach, 1996) while participants wear their normal aids for visual correction. The FrACT far visual acuity test has been successfully used with older adults with cognitive decline and dementia (Metzler-Baddeley et al., 2010). The test presents letters one by one in the middle of the monitor and requires participants to name them out loud as the experimenter enters the letters on the keyboard. Each letter is presented at high contrast and an adaptive procedure changes the letter size in order to estimate the smallest readable letter. Near visual acuity is measured using a standard Early Treatment Diabetic Retinopathy Study (ETDRS) chart. FIGS. 4B and 4C are graphical representations of data obtained from assessment of standardised vision measures of letter acuity at a close distance (40 cm) (FIG. 4C), and far acuity at 2 m (FIG. 4B) for subjects having NC and MCI.

### Stereoaculty

Stereoacuity refers to the smallest binocular disparity that an individual can accurately perceive in depth. Stereoacuity is measured with the standard Randot Stereo Test (Precision-Vision Inc., Woodstock, Illinois), which asks participants to indicate which of three circles on each row is closer to them than the others. Of the three circles presented, only one has a crossed disparity, which appears to stand forward from the other two when seen binocularly. FIG. 4A is a graphical representation of data obtained from assessment of standardised vision measures of stereoacuity for subjects having NC and MCI.

### Letter Contrast Senstivity

Contrast sensitivity refers to the minimum contrast that a participant can reliably detect at a range of spatial scales. Contrast sensitivity was measured using the computerized FrACT contrast sensitivity (Bach, 1996). The test presents a Landolt C target in one of four orientations (up, down, left or right) and asks the participant to indicate the direction where the opening of the C is located. FIG. 4D is a graphical representation of data obtained from assessment of standardised vision measures of letter contrast sensitivity for subjects having NC and MCI.

### Participants

Fifteen older adults (>55 years of age) with a confirmed diagnosis of mild cognitive impairment (MCI) were recruited among clients at Baycrest Centre for Geriatric Care. Individuals who had any other neurological disorder, history of stroke or transient ischemic attack, history of mental health disorders, or chemotherapy or radiation to the head were excluded. Fourteen older adults without a history of neurological diagnoses or MCI were recruited from the Rotman Research Institute's Participant Database to provide an age-and-gender-matched healthy control group. These participants were initially screened for signs of dementia using the Telephone Interview of Cognitive Status. All participants were administered the Montreal Cognitive Assessment (MoCA) during their first in-person visit to obtain a measure of their global cognition, and to serve as a benchmark screening test for MCI and Alzheimer's dementia. All participants provided written informed consent and/or assent prior to study enrolment. This study was approved by the Baycrest Research Ethics Board.

TABLE 1
Demographic information about the participants.

| | | | Education | |
|---|---|---|---|---|
| | n | Age (years) | (years) | MoCA |
| Group | (female) | M (SD) | M (SD) | M (SD) |
| Normal | 15 (9) | 73.07 (6.92) | 16.8 (2.31) | 26.87 (2.23) |
| cognition (NC) | | | | |
| Mild cognitive | 14 (6) | 74.78 (10.53) | 15.57 (1.87) | 22.69 (2.93) |
| impairment | | | | |
| (MCI) | | | | |

### Analysis Steps and Application to Assessment of Brain Health:

First, measures of behavioural performance (thresholds and accuracy) and/or EEG responses (event-related potentials, ERPs, and/or time-frequency spectra) obtained in each test can be examined separately to determine whether performance in that type of visual processing falls within normal limits for the age- and gender-group by comparing an individual's result to the distribution of performance from a normative group of age- and gender-matched adults with normal cognition.

Second, multivariate analyses, including multiple logistic regression, linear discriminant analysis, etc., can be performed to determine the optimal combination of behavioural and EEG markers across the tasks that results in the best classification of data from subjects with NC vs subjects with MCI. This model can then be used to determine the probability that a new subject has normal cognition or has cognitive impairment.

Third, evaluating performance in the same individual on several occasions across time can serve to monitor the change in performance or brain responses over time. The observation of a decline in one or multiple perceptual functions over time may signal early neurodegenerative processes. A normative database of longitudinal changes in adults with normal cognition may be used to compare the observed rate of change with the change expected with normal aging, for the purpose of identifying significant decline.

### Example Steps for the EEG Analysis:

CSV files containing the EEG data were imported into MATLAB and converted to an EEGLAB .SET file with appropriate channel locations. The data were then processed in the following order
1. Continuous EEG data were filtered using a fourth ordered, 0.1-15 Hz bandpass filter at a sampling rate of 256 Hz.
2. Stimulus-locked epochs were generated from 200 ms pre-stimulus to 500 ms post-stimulus.
3. The average voltage of the −200 to Oms pre-stimulus window was calculated, and subtracted from all values in that epoch.
4. Epochs with amplitudes below −50 pV or above +50 pV were identified and removed from the data set.
5. For each task, epochs were averaged together to form a single ERP per participant per task per channel/electrode.
6. For each participant and task, the data from the left and right temporoparietal channels were averaged.
7. Each participant's averaged temporoparietal ERP was inspected, and the latency of the largest peaks occurring in the following windows were automatically extracted:
   P1 window: positive peak between 50 to 150 ms
   N1/N170 window: negative peak between 80 to 250 ms

TABLE 2
Group means and standard deviations for the behavioural and electrophysiological
outcome measures for all four tests for older adults with normal cognition (NC) and with a
diagnosis of mild cognitive impairment (MCI). Statistical results for Welch t-test evaluating the
null hypothesis of no group differences is provided. P-values lower than a per-comparison alpha
level of 0.05 are bolded. Hedges' g provides a measure of effect size (>0.8 is a large effect).

| | | | | Mean | | | |
|---|---|---|---|---|---|---|---|
| | | NC | MCI | difference | Welch's t-test | p | |
| Task | Measure | M (SD) | M (SD) | Δ 95% CI | t, df | value | Hedges' g |
| Contours in | Threshold | 1.42 (0.2) | 1.14 | 0.28 [−0.46, | t (23.5) = −3.33 | 0.003 | −1.26 |
| clutter | | | (0.23) | −0.11] | | | |
| | P1 latency | 87.57 (11.95) | 102.73 | −15.17 [−4.04, | t (12.2) = 1.72 | 0.111 | 0.75 |
| | | | (25.72) | 34.38] | | | |
| | N1 latency | 142.25 | 176.95 | −34.7 [9.93, | t (16.3) = 2.97 | 0.009 | 1.26 |
| | | (22.62) | (30.71) | 59.47] | | | |
| Face | Accuracy | 0.81 (0.1) | 0.73 | 0.08 [−0.22, | t (11.8) = −1.24 | 0.238 | −0.57 |
| identification | | | (0.17) | 0.06] | | | |
| | P1 latency | 99.08 (9.77) | 115.02 | −15.94 [0.39, | t (11.3) = 2.25 | 0.045 | 1.03 |
| | | | (19.35) | 31.5] | | | |
| | N1 latency | 159.8 (14.77) | 174.48 | −14.68 [−5.52, | t (12.6) = 1.58 | 0.14 | 0.71 |
| | | | (24.55) | 34.88] | | | |
| Emotion | Accuracy | 0.89 (0.07) | 0.79 | 0.1 [−0.2, | t (13.9) = −2.49 | 0.026 | −1.08 |
| recognition in | | | (0.12) | −0.01] | | | |
| biological | P1 latency | 92.45 (15.65) | 95.31 | −2.86 [−21.07, | t (12.6) = 0.26 | 0.799 | 0.11 |
| walkers | | | (31.85) | 26.8] | | | |
| | N1 latency | 206.38 | 198.83 | 7.55 [−48.09, | t (19.7) = −0.39 | 0.701 | −0.16 |
| | | (47.19) | (43.75) | 32.99] | | | |

Central and Peripheral Divided Attention Test

| | | | | | | | |
|---|---|---|---|---|---|---|---|
| Central | Duration | 0.07 (0.01) | 0.08 | −0.01 [0, | t (16.2) = 1.53 | 0.145 | 0.59 |
| target, | threshold | | (0.02) | 0.02] | | | |
| focused | | | | | | | |
| attention | | | | | | | |
| Central | Duration | 0.06 (0.02) | 0.23 | −0.17 [0.02, | t (12.1) = 2.55 | 0.026 | 1.01 |
| target, | threshold | | (0.24) | 0.32] | | | |
| divided | | | | | | | |
| attention | | | | | | | |
| Peripheral | Duration | 0.15 (0.11) | 0.2 (0.13) | −0.05 [−0.04, | t (23.8) = 1.19 | 0.246 | 0.44 |
| target, | threshold | | | 0.15] | | | |
| focused | | | | | | | |
| attention | | | | | | | |
| Peripheral | Duration | 0.3 (0.18) | 0.41 | −0.1 [−0.03, | t (25.6) = 1.55 | 0.133 | 0.57 |
| target, | threshold | | (0.17) | 0.24] | | | |
| divided | | | | | | | |
| attention | | | | | | | |

Table 2 presents the results for all the behavioural and electrophysiological outcome measures across the four tasks separately for the NC and MCI groups. Electrophysiological data were analyzed by extracting the latency of the first positive peak (P1) and first negative peak (N1) in the ERP in each task.

Further, the bivariate correlations between each outcome measure and the Montreal Cognitive Assessment (MoCA) were examined, which provides a global measure of cognitive function and is used as a screening tool for mild cognitive impairment and Alzheimer's dementia.

### Contour in Clutter Task

FIG. 1C is a graphical representation of the relative density threshold values obtained for subjects having normal cognition (NC) and mild cognitive impairment (MCI), where the coloured circles represent individual subject data and the black dots and lines represent the group mean and standard deviation. FIG. 1D is a graphical representation of the association between density threshold values and MoCA scores obtained for subjects having NC and MCI. FIG. 1E is a graphical representation of group-averaged event-related potential (ERPs) obtained for subjects having NC and MCI. FIG. 1F a graphical representation of the association between the latency of the first negative component peak (N1) obtained for subjects having NC and MCI.

Participants with MCI showed worse performance (i.e., lower relative density thresholds) in the Contours in Clutter Test compared to normal controls (FIG. 1C), and this group difference was of a large effect size (p=0.003, g=−1.26). As shown in FIG. 1D, there was a significant positive correlation between MoCA scores and relative density threshold in the Contours in Clutter test (rho=0.46, p=0.003), indicating that individuals with better global cognition scores showed a better ability to discriminate the spiral contour in clutter.

Participants with MCI also showed significantly delayed responses in the EEG in response to the Contours in Clutter stimulus. As can be seen in Table 2, the latency of the N1 was higher in the MCI group compared to NC, and this effect also had a large effect size (p=0.009, g=1.26), (see FIG. 1E). The N1 latency in the Contours in Clutter task was also strongly negatively correlated with the MoCA (rho=−0.57, p=0.007) (FIG. 1F), indicating that better scores on the MoCA were seen in individuals with earlier peak N1 responses in this task.

### Face Identification Test

FIG. 2B is a graphical representation of mean accuracy (percent correct, PC) values obtained for subjects having NC and MCI. FIG. 2C is a graphical representation of the association between accuracy values and MoCA scores obtained for subjects having NC and MCI. FIG. 2D is a graphical representation of group-averaged ERPs obtained for subjects having NC and MCI. FIG. 2E is a graphical representation of the association between the latency of the first negative peak (N1) obtained for subjects having NC and MCI.

While there was no evidence that accuracy in the Face Identification Test was different in the NC and MCI groups (p=0.24, FIG. 2B), and accuracy was not correlated with the MoCA (rho =0.34, p=0.22, FIG. 2C), the P1 latency in the Face Identification task was significantly delayed in the MCI group relative to NC (p=0.045, g=1.03, see FIG. 2D), and both the P1 and N1 latencies were negatively associated with MoCA scores (e.g, P1: rho=−0.58, p=0.013, N1: rho=−0.52, p=0.02; FIG. 2E). Thus, while behavioural performance (accuracy) in the Face Identification test did not distinguish the two groups, measures derived from the EEG signals correlated with global cognitive status.

### Emotion Recognition in Biological Motion Test

FIG. 3B is a graphical representation of accuracy values obtained in the Emotion Recognition of Biological Motion test for subjects having NC and MCI, FIG. 3C is a graphical representation of the association between accuracy values in this test and MoCA scores obtained for subjects having NC and MCI. FIG. 3D is a graphical representation of ERP values obtained for subjects having NC and MCI. FIG. 3E is a graphical representation of the association between the latency of the peak of the first negative component (N1) obtained for subjects having NC and MCI.

Participants with MCI showed, on average, worse accuracy in the Emotion Recognition in Biological Motion Test (FIG. 3B), and this difference was statistically significant and had a large effect size (p=0.03, g=−1.08). As can be seen in FIG. 3C, accuracy in this task was also significantly correlated with the MoCA score (rho=0.54, p=0.01), with individuals with higher global cognition scores also showing better performance in identifying the emotion of biological walkers.

Stimuli in the Emotion Recognition in Biological Motion test did not generate pronounced P1 or N1 components in the current EEG recordings. Consequently, the latency of the P1 or N1 peaks did not show reliable differences between groups, and were not reliably associated with the MoCA. This lack of reliable ERP signals in this test may be due to a suboptimal placement of electrodes in this task.

### Central and Peripheral Divided Attention Test

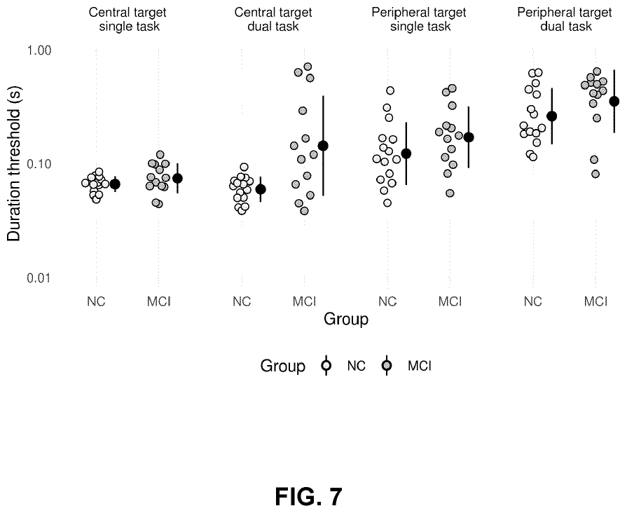

FIG. 7 is a graphical representation of the duration thresholds obtained for the central and peripheral targets under focused attention or divided attention conditions in the Central and Peripheral Divided Attention test. As can be seen, thresholds were worse for peripheral targets than central targets under both focused and divided attention conditions. Crucially, participants with MCI showed significantly higher thresholds than NC participants for the central target in the divided attention condition (p=0.026, g=1.01). This difference was not seen for the peripheral target in the divided attention condition (p=0.133). Therefore, the ability of the current method to evaluate the impact of dividing attention on both central and peripheral locations was important for revealing a deficit in the MCI group.

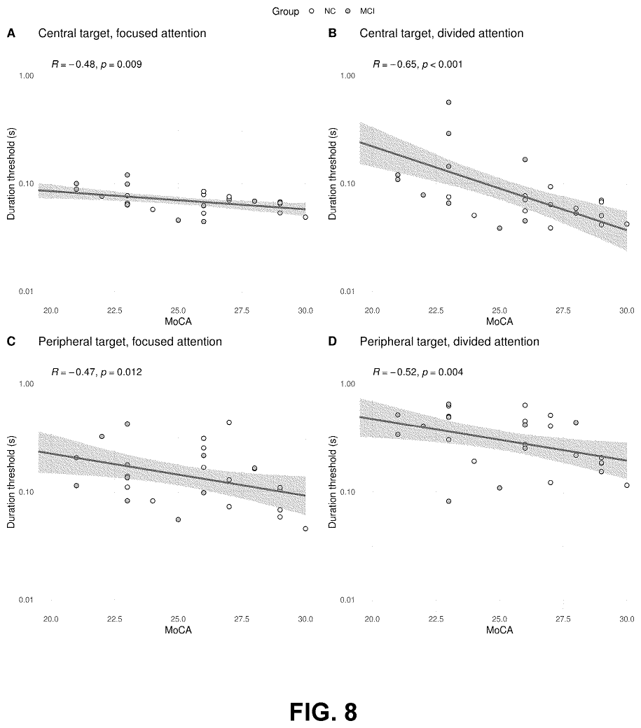

FIG. 8A-D is a graphical representation of the correlation between the MoCA scores and duration thresholds for central and peripheral targets, under focused and divided attention conditions. As can be seen, the strongest correlation was seen for the Central target, under divided attention (rho=−0.65, p<0.001, FIG. 8B), followed by the peripheral target, under divided attention (rho=−0.52, p=0.004, FIG. 8D). There were also significant correlations between MoCA and duration thresholds in the focused attention, central target (rho=−0.48, p=0.009, FIG. 8A) and peripheral target (−0.47, p=0.012, FIG. 8D). These results show that individuals with MCI require longer durations to extract relevant information from a visual scene under focused attention, and especially under divided attention.

### Example Multivariate Analysis

While measures obtained in any given test outlined above are able to distinguish between NC and MCI groups, it is also possible to analyze the pattern of results across multiple outcome variables in a multivariate analysis. A multivariate analysis combining behavioural and ERP measures from all the tests described above revealed a significant difference between the MCI and NC groups (Wilk's Lambda=0.27, F(7,12)=4.57, p=0.01), with an R² canonical=0.72 and 100% accurate classification in the current sample. This result indicates that the combination of performance across measures provides a strong ability to distinguish between mild cognitive impairment and normal cognition, with results from different tasks adding unique explanatory power to the overall classification of individuals in different groups greater than the ability of any one task to classify individuals. The current model can be used in the future to classify individuals with unknown cognitive status into either NC or MCI based on performance in behavioural and EEG measures.

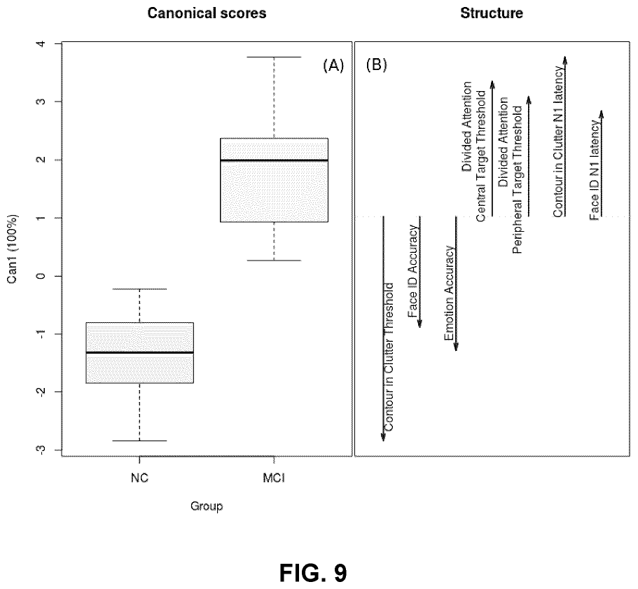

FIG. 9A is a graphical illustration of the linear discriminant weights obtained for each group, and FIG. 9B is a graphical illustration of the structure weights of each measure, which shows that the Contours in Clutter test measures made the largest contributions to the linear discriminant (threshold: −0.91, r²=0.82; N1 latency: 0.64, r²=0.41).

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

### CITED REFERENCES

Bach, M. (1996). The Freiburg Visual Acuity Test-Automatic Measurement of Visual Acuity. Optometry and Vision Science, 73(1), 49-53.
Fernandez, R., Kavcic, V., & Duffy, C. J. (2007). Neurophysiologic analyses of low- and high-level visual processing in Alzheimer disease. Neurology, 68(24), 2066-2076. https://doi.org/10.1212/01.wnl.0000264873.62313.81
Henry, J. D., Thompson, C., Rendell, P. G., Phillips, L. H., Carbert, J., Sachdev, P., & Brodaty, H. (2012). Perception of Biological Motion and Emotion in Mild Cognitive Impairment and Dementia. Journal of the International Neuropsychological Society, 18(05), 866-873. https://doi.org/10.1017/S1355617712000665
Hess, R. F., Hayes, A, & Field, D. J. (2003). Contour integration and cortical processing. Journal of Physiology, Paris, 97(2-3), 105-119. https://doi.org/10.1016/j.jphysparis.2003.09.013
Jastorff, J., De Wnter, F.-L., Van den Stock, J., Vandenberghe, R., Giese, M. A., & Vandenbulcke, M. (2016). Functional dissociation between anterior temporal lobe and inferior frontal gyrus in the processing of dynamic body expressions: Insights from behavioral variant frontotemporal dementia: Functional Dissociation Between ATL and IFG in Emotion Processing. Human Brain Mapping, 37(12), 4472-4486. https://doi.org/10.1002/hbm.23322
Lavallée, M. M., Gandini, D., Rouleau, I., Vallet, G T., Joannette, M., Kergoat, M.-J., Busigny, T., Rossion, B., & Joubert, S. (2016). A Qualitative Impairment in Face Perception in Alzheimer's Disease: Evidence from a Reduced Face Inversion Effect. Journal of Alzheimer's Disease, 51(4), 1225-1236. https://doi.org/10.3233/JAD-151027
Lockwood, C. T., Vaughn, W., & Duffy, C. J. (2018). Attentional ERPs distinguish aging and early Alzheimer's dementia. Neurobiology of Aging, 70, 51-58. https://doi.org/10.1016/j.neurobiolaging.2018.05.022
Konar, Y., Bennett, P. J., & Sekuler, A. B. (2013). Effects of aging on face identification and holistic face processing. Vision research, 88, 38-46.
Mapstone, M., Dickerson, K., & Duffy, C. J. (2008). Distinct mechanisms of impairment in cognitive ageing and Alzheimer's disease. Brain, 131(6), 1618-1629. https://doi.org/10.1093/brain/awn064
McIntosh, A. R., Sekuler, A. B., Penpeci, C., Rajah, M. N., Grady, C. L., Sekuler, R., & Bennett, P. J. (1999). Recruitment of unique neural systems to support visual memory in normal aging. Current Biology, 9(21), 1275-S2.
Metzler-Baddeley, C., Baddeley, R. J., Lovell, P. G., Laffan, A., & Jones, R. W. (2010). Visual impairments in dementia with Lewy bodies and posterior cortical atrophy. Neuropsychology, 24(1), 35-48. https://doi.org/10.1037/a0016834
Nasreddine, Z. S., Phillips, N. A., Bedirian, V., Charbonneau, S., Whitehead, V., Collin, I., Cummings, J. L., & Chertkow, H. (2005). The Montreal Cognitive Assessment, MoCA: A Brief Screening. Journal of the American Geriatrics Society, 695-699. https://doi.org/10.1111/j.1532-5415.2005.53221.x
Peuskens, H., Vanre, J., Verfaillie, K., & Orban, G. A. (2005). Specificity of regions processing biological motion. European Journal of Neuroscience, 21(10), 2864-2875. https://doi.org/10.1111/j.1460-9568.2005.04106.x
Pilz, K. S., Bennett, P. J., & Sekuler, A. B. (2010). Effects of aging on biological motion discrimination. Vision Research, 50(2), 211-219. https://doi.org/10.1016/j.visres.2009.11.014
Porter, G., Wattam-Bell, J., Bayer, A., Haworth, J., Braddick, O., Atkinson, J., & Tales, A. (2017). Different trajectories of decline for global form and global motion processing in aging, mild cognitive impairment and Alzheimer's disease. Neurobiology of Aging, 56, 17-24. https://doi.org/10.1016/j.neurobiolaging.2017.03.004
Roudaia, E., Bennett, P. J., & Sekuler, A. B. (2013). Contour integration and aging: The effects of element spacing, orientation alignment and stimulus duration. Frontiers in Psychology, 4(June), 356. https://doi.org/10.3389/fpsyg.2013.00356
Sauer, J., Ffytche, D. H., Ballard, C., Brown, R. G, & Howard, R. (2006). Differences between Alzheimer's disease and dementia with Lewy bodies: An fMRI study of task-related brain activity. Brain, 129(7), 1780-1788. https://doi.org/10.1093/brain/awl102
Sekuler, A. B., Bennett, P. J., Mamelak, M. A. (2000). Effects of aging on the useful field of view. Experimental aging research, 26(2), 103-120.
Spencer, J. M. Y., Sekuler, A. B., Bennett, P. J., Giese, M. A., & Pilz, K. S. (2016). Effects of aging on identifying emotions conveyed by point-light walkers. Psychology and Aging, 31(1), 126-138. https://doi.org/10.1037/a0040009
Troyer, A. K., Rowe, G, Murphy, K. J., Levine, B., Leach, L., & Hasher, L. (2014). Development and evaluation of a self-administered on-line test of memory and attention for middle-aged and older adults. Frontiers in Aging Neuroscience, 6. https://doi.org/10.3389/fnagi.2014.00335
Uhlhaas, P. J., Pantel, J., Lanfermann, H., Prvulovic, D., Haenschel, C., Maurer, K., & Linden, D. E. J. (2008). Visual perceptual organization deficits in Alzheimer's dementia. Dementia and Geriatric Cognitive Disorders, 25(5), 465-475. https://doi.org/10.1159/000125671
Volberg, G, & Greenlee, M. W. (2014). Brain networks supporting perceptual grouping and contour selection. 5(April), 1-17. https://doi.org/10.3389/fpsyg.2014.00264
Watson, A. B. (2017). QUEST+: A general multidimensional Bayesian adaptive psychometric method. Journal of Vision, 17(3), 10. https://doi.org/10.1167/17.3.10
Yamasaki, T., Goto, Y., Ohyagi, Y., Monji, A., Munetsuna, S., Minohara, M., Minohara, K., Kira, J., Kanba, S., & Tobimatsu, S. (2012). Selective Impairment of Optic Flow Perception in Amnestic Mild Cognitive Impairment: Evidence From Event-Related Potentials. Journal of Alzheimer's Disease, 28(3), 695-708. https://doi.org/10.3233/JAD-2011-110167
Yamasaki, T., Horie, S., Ohyagi, Y., Tanaka, E., Nakamura, N., Goto, Y., Kanba, S., Kira, J., & Tobimatsu, S. (2016). A Potential VEP Biomarker for Mild Cognitive Impairment: Evidence from Selective Visual Deficit of Higher-Level Dorsal Pathway. Journal of Alzheiner's Disease, 53(2), 661-676. https://doi.org/10.3233/JAD-150939

1.-44. (canceled)

45. A computer implemented method for assessing brain health in a subject, comprising the steps of:
administering one or more visual perception tests to the subject, wherein each visual perception test comprises presentation of a series of visual stimuli to the subject, wherein the series of visual stimuli comprises an initial fixation point followed by a sequence of one or more target images, and wherein the sequence of target images in each visual perception test is designed to measure visual perceptual and/or attentional functions without relying on memory or language;
obtaining:
a measurement of behavioural responses during administration of the one or more visual perception tests and extracting indices of perceptual ability for each test from the behavioural responses, wherein the behavioural responses relate to identification of a characteristic of the one or more target images in the series of visual stimuli, and wherein the indices of perceptual ability relate to performance threshold and/or performance accuracy; and
a simultaneous measurement of electrophysiological responses of the subject using electroencephalography (EEG) sensors during administration of the one or more visual perception tests and a time-based correlation of events occurring during the tests with the obtained electrophysiological responses, and extracting indices of electrophysiological responses, wherein the indices of electrophysiological responses are derived from raw EEG signals, event-related potentials and/or time-frequency spectra; and
comparing the obtained perceptual ability indices, the obtained electrophysiological indices, or both, from one or more of the tests, with a comparative data set to identify deviations from normative performance in any measure or combinations of measures to create a combined index that provides an assessment of brain health in the subject;
wherein the comparative data set is:
a normative data set of healthy older adults with normal cognition to identify deviations from normative performance in any measure or combinations of measures to provide an assessment of brain health in the subject; or
data obtained in one or more tests previously administered to the subject, to identify changes in performance in any measure or combinations of measures to provide an assessment of changes in the brain health state in the subject.

46. The method of claim 45, wherein the one or more visual perception tests are selected from a Contours in Clutter test, a Face Identification test, an Emotion Recognition in Biological Motion test, and a Central and Peripheral Divided Attention test.

47. The method of claim 45, wherein the assessment of brain health comprises determination of mild cognitive impairment.

48. The method of claim 45, wherein the assessment of brain health comprises determination of a neurodegenerative disorder.

49. The method of claim 48, wherein the neurodegenerative disorder is classical Alzheimer's disease, Alzheimer's disease with posterior cortical atrophy, Lewy Body disease, frontotemporal dementia, or vascular dementia.

50. The method of claim 45, wherein the assessment of brain health comprises an assessment of changes in brain health over a period of time to track progression of disease, to track effects of treatments or behavioural interventions, or to predict changes in perceptual and cognitive status.

51. The method of claim 45, wherein the measure of the electrophysiological responses is carried out using a portable EEG device.

52. The method of claim 45, wherein each visual perception test comprises one or more trials, wherein each trial comprises the steps of:
a. presenting the visual stimulus to the subject;
b. recording the behavioural response by the subject and/or the EEG response to brain activity during the trial.

53. The method of claim 52, wherein the one or more visual perception tests includes the Contours in Clutter test wherein the visual stimulus comprises a sequence of images, each image comprising an array of oriented visual stimulus elements in which a subset of stimulus elements forms a target contour through alignment of neighboring elements;
wherein the visibility of the target contour is varied between trials by altering the display characteristics to measure an ability of the subject to perceive the target contour within a cluttered background; and
wherein the subject identifies an element of the target contour to exhibit knowledge of the shape and/or location of the definable contour.

54. The method of claim 53, wherein a measure of the subject's tolerance for clutter in performing the task and/or the electrophysiological response is compared with the comparative data set, in isolation or in combination with other measures.

55. The method of claim 53, wherein the degree of clutter is varied across trials to determine the degree of clutter that yields a specified level of accuracy.

56. The method of claim 53, wherein the target contour is a spiral-shaped contour formed through alignment of the orientations of the subset of stimulus elements along a spiral path.

57. The method of claim 56, wherein the subject is prompted to report whether the tail of the spiral-shaped contour is on the left or right side of the image.

58. The method of claim 52, wherein the one or more visual perception tests includes the Contours in Clutter test,
wherein the visual stimulus comprises a sequence of images, each image comprising an array of randomly oriented elements comprising background elements and a subset of contour elements arranged to form a spiral-shaped contour through alignment of the orientations of the subset of contour elements along the spiral path;
wherein the relative density of the background and contour elements used in each image in the sequence of images is varied to determine the degree of relative density that yields a specified level of accuracy, and
wherein the subject is prompted to report whether the tail of the spiral-shaped contour was on the left or right side of the image.

59. The method of claim 58, wherein the degree of relative density to yield a specific level of accuracy and/or the electrophysiological response is compared with the comparative data set, in isolation or in combination with other measures.

60. The method of claim 52, wherein the one or more visual perception test includes the Face Identification test,
wherein the visual stimulus comprises a sequence of images, the sequence of images comprising a first image and a second image, wherein the first image is a test face, and the second image comprises two or more probe faces, wherein one of the probe faces is of the same identity as the test face; and
wherein the subject identifies which probe face matches the identity of the test face;

61. The method of claim 60, wherein a measure of the ability of the subject to identify face identity when performing the task and/or the electrophysiological response is compared with the comparative data set, in isolation or in combination with other measures.

62. The method of claim 60, wherein the difficulty of the task is varied across trials by altering the display characteristics to measure the ability of the subject to identify face identity under different stimulus conditions.

63. The method of claim 62, wherein the display characteristics are altered by varying one or more of the stimulus duration, stimulus contrast, degree of added visual noise, manipulation of facial appearance, facial viewpoint, facial orientation, addition of a masking stimulus before or after presentation of faces.

64. The method of claim 52, wherein the one or more visual perception tests includes the Face Identification test,
wherein the visual stimulus comprises a sequence of images, the sequence of images comprising a first image and a second image, wherein the first image is a test face facing in a first direction, and the second image comprises two or more probe faces, wherein each probe face is facing in a second direction different from the first direction, and wherein one of the probe faces shows the same person as the test face; and
wherein the subject is prompted to indicate which probe face matches the identity of the test face.

65. The method of claim 64, wherein a measure of the ability of the subject to identify face identity when performing the task and/or the electrophysiological response is compared with the comparative data set, in isolation or in combination with other measures.

66. The method of claim 52, wherein the one or more visual perception tests includes the Emotion Recognition in Biological Motion test,
wherein the visual stimulus comprises a sequence of animated images, each animated image comprising depictions of a human walker conveying an emotional state through its gait; and
wherein the subject indicates which emotion the walker portrayed.

67. The method of claim 66, wherein a measure of the ability of the subject to identify emotion of the walker when performing the task and/or the electrophysiological response is compared with the comparative data set, in isolation or in combination with other measures.

68. The method of claim 66, wherein the difficulty of the task is varied across trials by altering the display characteristics to measure the ability of the subject to identify the emotion of the walker under different stimulus conditions.

69. The method of claim 68, wherein the display characteristics are altered by varying one or more of the stimulus duration, stimulus contrast, degree of added visual noise, manipulation of walker appearance, walker viewpoint, walker orientation, addition of a masking stimulus before or after presentation of walkers.

70. The method of claim 52, wherein the one or more visual perception tests includes the Emotion Recognition in Biological Motion test,
wherein the visual stimulus comprises a sequence of animated images, each animated image comprising depictions of point light walker shown for a single gait cycle, wherein the gait is happy, angry, or sad;
wherein the subject is prompted to report which emotion the point light walker portrayed.

71. The method of claim 70, wherein a measure of the ability of the subject to identify emotion of the walker when performing the task and/or the electrophysiological response is compared with the comparative data set, in isolation or in combination with other measures.

72. The method of claim 52, wherein the one or more visual perception tests includes the Central and Peripheral Divided Attention test,
wherein the visual stimulus comprises a sequence of images, each image comprising one or more central target(s) and/or one or more peripheral target(s) presented in a number of possible locations;
wherein the subject reports target attributes selected from the identity of the central target(s), or one or more of the radial direction, location, identity of the peripheral target(s), or both central and peripheral target attributes.

73. The method of claim 72, wherein a measure of the ability of the subject to identify the target attributes when performing the task and/or the electrophysiological response is compared with the comparative data set, in isolation or in combination with other measures.

74. The method of claim 72, wherein the difficulty of the task is varied across trials by altering the display characteristics to measure the ability of the subject to identify target attributes in central and/peripheral locations.

75. The method of claim 74, wherein the display characteristics are altered by varying one or more of the stimulus duration, stimulus contrast, spatial distribution of target locations, degree of added visual noise, numbers of central and/or peripheral targets, similarity among central and/or peripheral targets, addition of a masking stimulus before or after presentation of targets.

76. The method of claim 52, wherein the one or more visual perception tests includes the Central and Peripheral Divided Attention test,
wherein the visual stimulus comprises a sequence of images, each image comprising a central target and/or a peripheral target presented in one of a number of possible locations;
wherein the subject is cued in advance of a trial whether the trial will comprise a central target, a peripheral target, or both targets;
wherein the duration of target exposure is varied across trials to determine a target exposure duration that yields a specified accuracy level; and
wherein, the subject is asked to report the identity of the central target, or to report the radial direction of the peripheral target, or to report the attributes of both targets.

77. The method of claim 76,
wherein, at the start of a trial, a preparatory screen comprising a central fixation point and 16 circular outlines is presented to the subject, wherein the central fixation point indicates the location of the central target and the circular outlines indicate the potential locations of the peripheral target; and
wherein, if a subsequent central target appears, the subsequent central target is selected from among a set of letters; and
wherein, if a subsequent peripheral target appears, the subsequent peripheral target is a filled circle within one of the 16 circular outlines; and
wherein, subsequent to presentation of the central and/or peripheral target, all potential target locations are covered with a masking stimulus.

78. The method of claim 76, wherein a measure of the ability of the subject to identify the target attributes when performing the task and/or the electrophysiological response is
compared with the comparative data set, in isolation or in combination with other measures.
